(19) United States
(12) Patent Application Publication
(10) Pub. No.: US 2005/0267415 A1
(43) Pub. Date: Dec. 1, 2005

(54) Medical devices and methods of use

(75) Inventor: Steven L. Jacques, Westford, MA (US)

Correspondence Address: HOLLAND & HART, LLP, 555 17TH STREET, SUITE 3200, DENVER, CO, 80201, US

(73) Assignee: C. R. Bard, Inc.

(21) Appl. No.: 11/127,662

(22) Filed: May 12, 2005

Related U.S. Application Data
(60) Provisional application No. 60/571,420, filed on May 14, 2004.

(51) Int. Cl. A61M 5/32

(52) U.S. Cl. 604/175

(57) ABSTRACT

A medical device for creating a fistula in a patient has a catheter including a first lumen and a second lumen for forming a fistula inside a body organ of a patient is disclosed. The second lumen may be capable of fluid communication with an internal bolster, wherein the internal bolster is attachable to the catheter near the distal end and is at least partially filled with a liquid. An external bolster may be adjustably attachable to the catheter outside the patient. Methods of use of medical devices according to the present invention are also disclosed. In one aspect of a method of use, at least a portion of the liquid within an internal bolster may be removed. Such a process may substantially collapse the internal bolster for removal of the medical device.

### RELATED APPLICATION

This application claims the benefit of U.S. Provisional Patent Application No. 60/571,420, filed May 14, 2004, the disclosure of which is incorporated, in its entirety, by this reference.

### BACKGROUND OF THE INVENTION

Patients may develop conditions that prevent them from eating or swallowing normally. Additionally, patients may develop conditions that prevent the proper drainage of certain organs, such as the bladder. In such cases, it may be beneficial to create an artificial passage, or fistula, between the affected organ and the exterior of the patient's body. In one example, when a patient's ability to eat or swallow is affected, a percutaneous endoscopic gastrostomy (“PEG”) device may be employed for providing nutrient to the patient through a fistula formed between the stomach and the exterior of the patient. PEG devices are often commonly referred to as “feeding tubes.” PEG devices may be initially placed via the esophagus using an endoscope. The initial placement PEG device is guided down the patient's esophagus, into the stomach, and passed out a surgical opening in the abdomen. Internal and external bolsters secure the PEG device to the stomach wall and the exterior surface of the patient's body, establishing a direct passage into the patient's stomach, through which nourishment may be provided. However, PEG devices need to be changed periodically. Accordingly, a replacement device may be inserted into the stomach via the established stoma in the patient's abdomen that was formed during the initial placement procedure. Thus, the patient may avoid another endoscopic procedure via the esophagus, because a replacement device may be inserted through the existing stoma. Accordingly, such devices that are initially placed or installed may be generally described as “initial placement” devices and devices that are subsequently placed or installed may be described as “replacement” devices.

Because installation techniques for initial placement devices and replacement devices may be different, such devices may be structured differently (i.e., with respect to its respective process for installation). Many conventional initial placement devices include a soft silicone dome as the internal bolster, while several of the replacement devices currently marketed incorporate a balloon as the internal bolster. A conventional initial placement PEG device is described, for example, in U.S. Pat. No. 5,910,128 to Quinn, the disclosure of which is incorporated, in its entirety, by this reference.

In devices using an air-filled internal bolster, the air in the bolster may compress very easily. Consequently, the retention forces are not optimal and some patients may be able to remove the device from the stoma. Additionally, if the feeding tube is held incorrectly during traction removal, the air lumen may be pinched off, which may result in undesirably high removal forces. Also, during the removal step, the bolster wall may fold over the airway notch as the device is pulled through the stoma, sealing off the air lumen and preventing air evacuation and resulting in undesirably high removal forces.

### SUMMARY OF THE INVENTION

One aspect of the present invention relates to a medical device including an internal bolster that is at least partially expanded or filled with a liquid such as, for instance, a sterile saline or a similar liquid. Such a configuration may result in relatively high retention force and a relatively low removal force for such a medical device. In addition, such a configuration may allow the internal bolster to be reduced in diameter, such that the profile of the installed device is smaller than that of a conventional air-filled internal bolster.

Another aspect of the present invention relates to methods of use of a medical device according to the present invention. For example, the present invention contemplates a method for removing a medical device from a patient. Particularly, a medical device may be positioned so as to form a fistula within an organ of a patient, wherein the medical device includes an internal bolster at least partially filled with a liquid, wherein the internal bolster is configured for retaining at least a portion of the medical device within the organ of the patient. Further, at least a portion of the liquid may be removed from the internal bolster and the medical device may be removed from the patient. In another method of use, the present invention contemplates that a catheter may be provided, the catheter including: a first lumen and a second lumen, the catheter including a distal end and a proximal end, and an internal bolster attachable to the catheter near the distal end thereof which is capable of fluid communication with the second lumen. Also, the proximal end of the catheter may include a pull device configured for facilitating pulling the proximal end of the catheter through a stoma formed in an organ of a patient and the first lumen is configured for providing a fistula to an organ of a patient. Further, the distal end of the catheter may be inserted through an esophagus and into a stomach of a patient and the distal end of the catheter may be pulled through a stoma formed in a wall of the stomach so as to position the internal bolster proximate thereto. A portion of the catheter positioned external to the patient may be severed and the internal bolster may be at least partially expanded with a liquid so as to facilitate retention of the catheter within the patient.

Another aspect of the present invention relates to a medical device. In one embodiment, a medical device may include a catheter including a first lumen and a second lumen, the catheter including a distal end and a proximal end, wherein the first lumen is sized and configured for providing a fistula to an organ of a patient and the second lumen is capable of fluid communication with the internal bolster. Further, an internal bolster may be attachable to the catheter near the distal end thereof and fillable with a volume of liquid. Additionally, the first lumen may be configured for providing a fistula to an organ of a patient and the second lumen may be configured for fluid communication with the internal bolster. In another embodiment of a medical device according to the present invention, the medical device may comprise a catheter including a first lumen and a second lumen, the catheter including a distal end and a proximal end, wherein the proximal end of the catheter includes a pull device configured for facilitating pulling the proximal end of the catheter through a stoma formed in a stomach of a patient. The catheter may include an internal bolster attachable thereto near the distal end thereof. Also, the first lumen may be configured for providing a fistula to an organ of a patient and the second lumen may be configured for fluid communication with the internal bolster.

Such medical devices including an internal bolster may generate superior retention forces such that there is a reduced likelihood of inadvertent removal of the medical device by a patient pulling on the proximal end. On the other hand, a medical device according to the present invention may exhibit relatively low removal forces when the internal bolster has been substantially collapsed. In some embodiments of the present invention, the medical device may be used as an initial placement gastrostomy device for creating a fistula with a patient's stomach. Other embodiments may use the medical device to provide artificial access to a patient's bladder for the drainage of body fluids. It should be understood that the present invention could be adapted to create any number of fistulae in a patient's body for access to various organs thereof.

### BRIEF DESCRIPTION OF THE DRAWINGS

Further features of the invention, its nature, and various advantages will be more apparent from the following detailed description and the accompanying drawings, wherein like reference characters represent like elements throughout, and in which:

FIG. 1 shows a schematic perspective view of an embodiment of an apparatus in accordance with the present invention;

FIG. 2 shows a cross-sectional view of the apparatus shown in FIG. 1 taken with respect to line 2-2 of FIG. 1;

FIG. 3 shows a schematic view of the apparatus illustrated in FIG. 1 positioned within a human body;

FIG. 4 shows a perspective view of an embodiment of an apparatus in accordance with the present invention;

FIG. 5 shows an enlarged perspective view of the apparatus illustrated in FIG. 4;

FIG. 6 shows a cross-sectional view of the apparatus shown in FIG. 5 taken with respect to line 5-5;

FIG. 7 shows a perspective view of an embodiment of an apparatus in accordance with the present invention;

FIG. 8 shows a perspective view of an apparatus according to the present invention;

FIG. 9 is an enlarged side cross-sectional view of the apparatus illustrated in FIG. 8;

FIG. 10 shows an exploded assembly view of an embodiment of an apparatus in accordance with the present invention;

FIGS. 11-13 show schematic views of the apparatus illustrated in FIG. 10 during assembly procedures therefor;

FIG. 14 shows an exploded assembly view of an embodiment of an apparatus in accordance with the present invention;

FIG. 15 shows an enlarged perspective view of a portion of the apparatus illustrated in FIG. 14;

FIG. 16 shows a side cross-sectional view of the apparatus illustrated in FIG. 14;

FIG. 17 shows a schematic, enlarged view of the apparatus illustrated in FIG. 3 positioned within a human body;

FIG. 18 shows a schematic, enlarged view of the apparatus illustrated in FIG. 3 positioned within a human body including a syringe in communication with the internal bolster of the apparatus; and

FIG. 19 shows a schematic, enlarged view of the apparatus illustrated in FIG. 3 positioned within a human body wherein the internal bolster is collapsed.

### DETAILED DESCRIPTION OF THE INVENTION

Because the present invention has a number of different applications, each of which may warrant some modifications of such parameters as instrument size and shape, it is believed best to describe certain aspects of the invention with reference to relatively generic schematic drawings. To keep the discussion from becoming too abstract, however, and as an aid to better comprehension and appreciation of the invention, references will be made to specific uses of the invention. For example, reference will be made to use of the invention to provide a fistula to a patient's stomach. It is emphasized, however, that this is only one of many possible applications of apparatuses and methods according to the present invention.

One embodiment of a PEG apparatus according to the present invention may be utilized for initial placement of a PEG device within a patient's stomach (i.e., to provide a fistula to a patient's stomach). More particularly, a procedure may begin by inserting an initial placement PEG device including an internal bolster and an endoscope into a patient's esophagus. The PEG device may be guided down the esophagus, into the stomach, through the stomach wall, and through the patient's abdomen via a surgical opening. In accordance with one initial placement embodiment of the present invention, the internal bolster may be deflated prior to the beginning of the endoscopic procedure. As described below, the internal bolster may be expanded by using a luer-tip needle, a syringe, or a similar device to introduce a fluid (e.g., a gas or a liquid) therein. Another embodiment of a PEG apparatus according to the present invention may be utilized for replacing another PEG device, wherein the replacement PEG apparatus according to the present invention may be placed through a stoma created in a patient's abdomen. While initial placement embodiments may be installed with the internal bolster in its deflated state, the replacement embodiments may be placed with the internal bolster in an at least partially expanded state (i.e., intermediates between fully expanded and deflated or collapsed) or a deflated or collapsed state, without limitation.

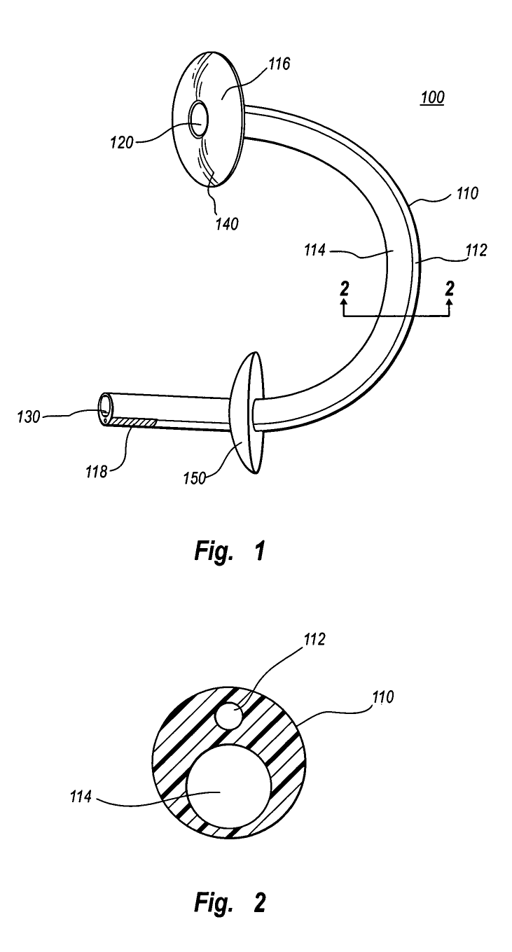

One embodiment of a medical device 100, which is shown as an initial placement PEG device, is shown in FIG. 1. Medical device 100 may include catheter 110 including distal opening 120 and proximal opening 130. Catheter 110 may incorporate two lumens, namely, a bolster control lumen 112 and a fistulous lumen 114. The distal end of bolster control lumen 112 may include a bolster control port 116. Bolster control port 116 may be structured for allowing or controlling fluid communication with internal bolster 140. For example, bolster control port 116 may comprise a valve or other flow control device as known in the art. An internal bolster 140 may be located at the distal end of catheter 110 and may be an integral part of catheter 110 or may be attached to catheter 110 by some other means (e.g., adhesive, chemically bonded, welded, etc.). In one embodiment, internal bolster 140 acts as a balloon which may be inflated or expanded with a fluid (e.g., air, water, or a sterile saline solution). If desired, the proximal end of bolster control lumen 112 may be occluded by plug 118 or may be otherwise sealed or occluded as known in the art. Plug 118 may be constructed of a material that is penetrable by a needle, but which seals the perforation of the needle upon removal thereof. Medical device 100 may also include an external bolster 150. External bolster 150 may be movable along catheter 110 and may be selectively secured thereto or positioned thereabout such that catheter 110 may be positioned within a patient's stomach with internal bolster 140 engaging or contacting against the stomach wall of the patient and the external bolster 150 may be positioned so as to contact snugly and securely against the external region of a patient's abdomen. Thus, external bolster 150 may be moved or adjusted along the length of catheter 110 to achieve a desired placement and suitable fit.

FIG. 2 shows a cross-sectional view of catheter 110. Catheter 110 may be constructed of a material that is suitable for use in medical devices. For example, catheter 110 may be constructed of any polymer, plastic, or any suitable material as known in the art, without limitation. In one embodiment, catheter 110 may comprise polyurethane. As shown in FIG. 2, catheter 110 may be a dual-lumen catheter, comprising bolster control lumen 112 and fistulous lumen 114. Further, as shown in FIG. 2, bolster control lumen 112 may be substantially smaller than fistulous lumen 114.

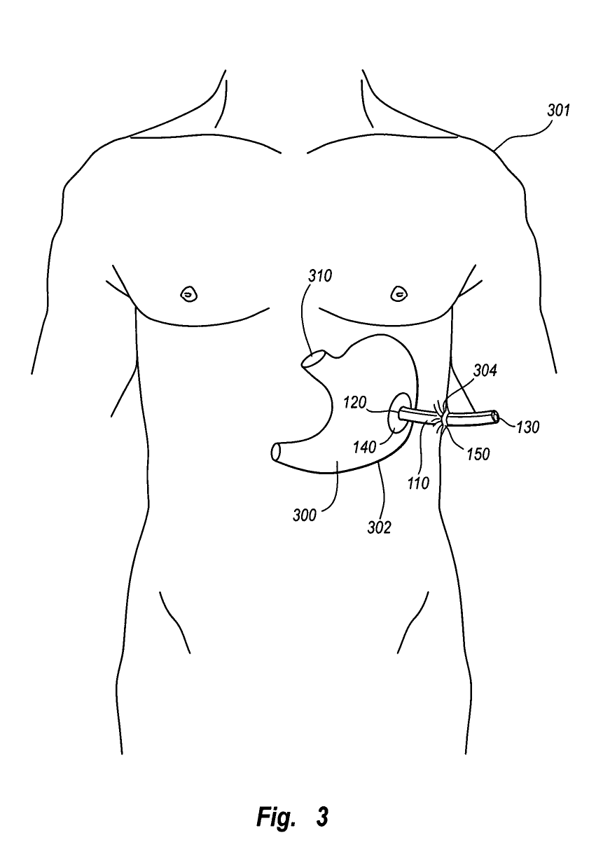

FIG. 3 shows a schematic view of medical device 100 positioned within patient 301. In one embodiment, medical device 100 (e.g., an initial placement PEG device) may be positioned within a patient 301 via the esophagus 310. Medical device 100 may exit the body through a stoma surgically created at an abdominal surface 304. Thus, a fistula between the stomach 300 and the exterior of a patient's body may be formed by a fistulous lumen 114 (FIG. 2) of catheter 110. In further detail, catheter 110, and more particularly fistulous lumen 114 (FIG. 2) thereof, may form a passage between stomach 300 and abdominal surface 304 via distal opening 120 and proximal opening 130. Internal bolster 140 and external bolster 150 may be positioned against stomach wall 302 and abdominal surface 304, respectively. Internal bolster 140 and external bolster 150 may be structured for retaining the medical device 100 within patient 301. Medical device 100 may also include an anti-reflux valve and a plug to seal proximal opening 130 when medical device 100 is not being used as a conduit for communicating nutrients or medication to the stomach 300 of the patient 301.

During initial placement or implantation of medical device 100 (i.e., not replacing another feeding tube device), the details of the endoscopic placement procedure may encompass any such procedures as known in the art. For example, for ease of movement down esophagus 310, internal bolster 140, if at least partially inflated or expanded, may be elongated or otherwise deformed (e.g., by using a stylet or other deformation mechanism) as known in the art. Once medical device 100 is suitably placed and internal bolster 140 is positioned at a selected location along stomach wall 302, the internal bolster 140 may be returned to its original (i.e., not deformed) shape (e.g., by removing the stylet). In another embodiment, medical device 100 may be endoscopically placed with internal bolster 140 in a deflated state. Once internal bolster 140 is positioned at a selected location along stomach wall 302, internal bolster 140 may be inflated or expanded by communicating fluid (e.g., sterile saline) into the bolster 140 via the bolster control lumen 112.

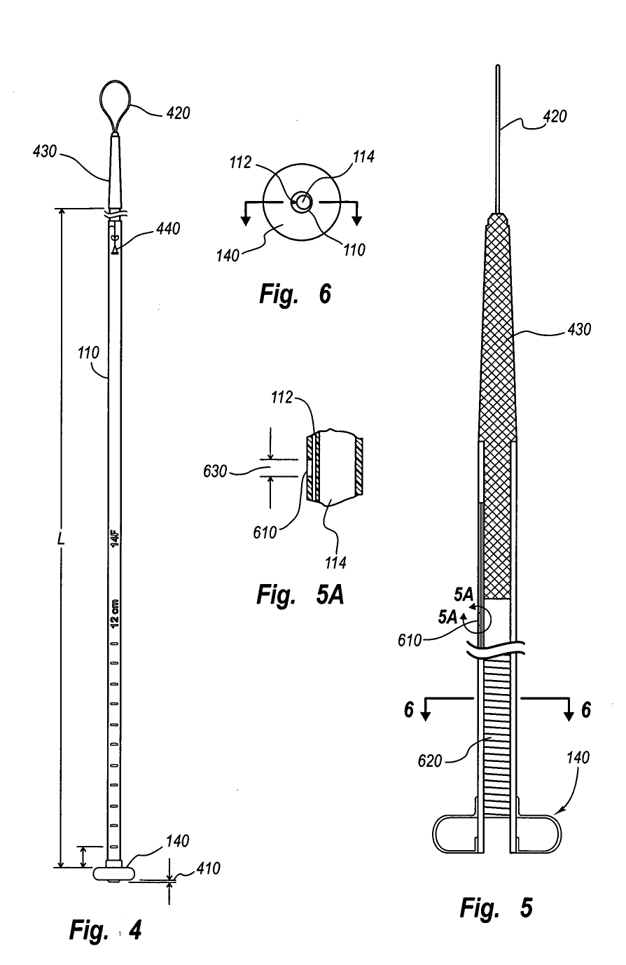

FIG. 4 shows medical device 100 prepared for an initial placement endoscopic procedure. Catheter 110 has an initial length L and is attached to pull device 430. Pull device 430 may include a snare loop 420. Catheter 110 may be marked with cut-line 440 for cutting reference after placement of the device. In one embodiment, catheter 110 may not extend beyond the distal surface of internal bolster 140 by more than dimension 410. For example, dimension 410 may be about 0.000 to about 0.012 inches.

FIG. 5 shows the arrangement of bolster control lumen 112 and fistulous lumen 114 within catheter 110 and with respect to internal bolster 140 in one embodiment of the present invention. To reduce the size of internal bolster 140 during placement of medical device 100, bolster control lumen 112 may include aperture 610, as shown in FIG. 6. Aperture 610 may allow fluid (e.g., air or liquid) to escape from internal bolster 140 when it meets resistance as it passes down the esophagus during the initial placement procedure. Thus, aperture 610 may be configured to release pressure from internal bolster 140. Aperture 610 may have a dimension 630 as shown in FIG. 6. More specifically, dimension 630 may be about 0.03 inches. In another embodiment, instead of aperture 610 or in addition thereto, a one-way valve may be in communication with bolster control lumen 112 and may be configured for releasing a fluid (e.g., a gas, a liquid, or both) from within bolster control lumen 112 in response to a magnitude of pressure therein exceeding a selected maximum magnitude of pressure. Medical device 100 may be reinforced along a portion of its length with reinforcing coil 620 or some other reinforcing member as known in the art. Allowing air to evacuate from internal bolster 140 may help maintain internal bolster 140 in a collapsed state and prevent resistance by trapped air. Collapsed internal bolster 140 may become softer and more pliable and have a reduced size or profile for facilitating placement thereof through an esophagus. Patients may benefit from such a configuration, because such a medical device 100 may provide ease of insertion, improved passage through the esophagus, improved passage in a smaller esophagus (e.g., neo-natal, pediatric, small children, or patients with a narrowed esophagus), and less trauma to an esophagus.

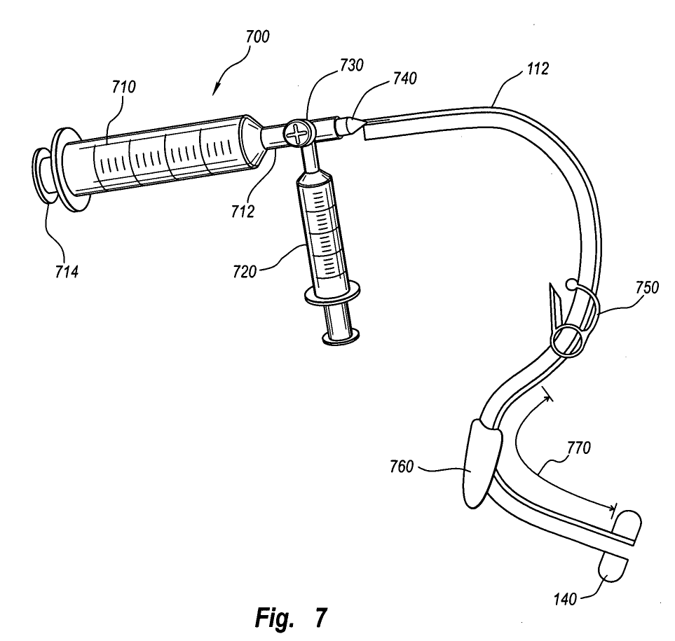

Known “pull” or “guidewire” techniques may be used to endoscopically place medical device 100. Once pull device 430 has exited the abdominal wall, medical device 100 may be pulled until reinforced portion 770 is exposed. At this point, at least a portion of catheter 110 external to the patient (e.g., substantially at cut-line 440) may be removed or severed from the remaining catheter 110. Subsequent to severing catheter 110, an external bolster 150 may be positioned upon or along catheter 110. FIG. 7 shows a so-called “90°” bolster 760. However, a linear bolster, such as linear bolster 150 as shown in FIG. 3 may be used, if desired, without limitation. External bolster 760 may be positioned along the remaining length of catheter 110 until it is located over reinforced portion 770. Further clamp device 750 may be coupled (i.e., positioned upon or attachable) to catheter 110. For example, clamp device 750 may be positioned just above reinforced portion 770. Once medical device 100 is initially placed with deflated internal bolster 140 and external bolster 760 and clamp device 750 have been placed, the physician may perform a procedure for at least partially expanding the internal bolster 140.

Specifically, FIG. 7 illustrates equipment 700 which may include syringe 710, syringe 720, stopcock 730, and luer-tip needle 740. Stopcock 730 may comprise a so-called “3-way” stopcock as known in the art and may be operably coupled to syringe 710. Additionally, luer-tip needle 740 may be capable of fluid communication with stopcock 730. Plunger 712 may be positioned as shown with plunger control 714. Syringe 720 is filled with the appropriate amount of liquid (e.g., sterile saline) for the size of medical device 100, which may be manufactured, for example, in 12, 14, 16, 18, 20, or 24 Fr. sizes (e.g., 2 cc's may be used for a 14 Fr. device). Air may be purged from syringe 720 prior to filling it with the appropriate amount of liquid. Subsequent to purging air from syringe 720, syringe 720 may be coupled to stopcock 730. Additionally, luer-tip needle 740 may be inserted into bolster control lumen 112 of medical device 100 until it contacts the luer hub. Stopcock 730 may be positioned such that syringe 710 is in fluid communication with luer-tip needle 740. Plunger control 714 of syringe 710 may be actuated to evacuate air, liquid, or both from internal bolster 140 that may exist after the placement procedure is performed. Put another way, syringe 710 may be employed for generating a vacuum within bolster control lumen 112 and internal bolster 140. Of course, in another embodiment, a vacuum pump or other vacuum generating device may be employed for generating a vacuum within bolster control lumen 112 and internal bolster 140. It may be further noted that aperture 610 may be removed from medical device 100 with the portion of catheter 110 severed therefrom (i.e., proximally from cut-line 440 (FIG. 4)). Subsequently, clamp device 750 may be closed, and stopcock 730 may be operated such that fluid communication is prevented between syringe 710 and luer-tip needle 740 and fluid communication is provided between syringe 720 and bolster control lumen 112. Clamp device 750 may be reopened and syringe 720 may be operated to introduce liquid within internal bolster 140 via bolster control lumen 112 until a selected volume of liquid (e.g., saline or sterile water) in syringe 720 has been transferred from syringe 720 to internal bolster 140 for expansion or inflation thereof. Clamp device 750 may be again closed, and equipment 700 may be removed from the proximal end of medical device 100. At this point, clamp device 750 may be positioned such that a feeding adaptor may be positioned within a proximal end of the remaining portion of the catheter 110.

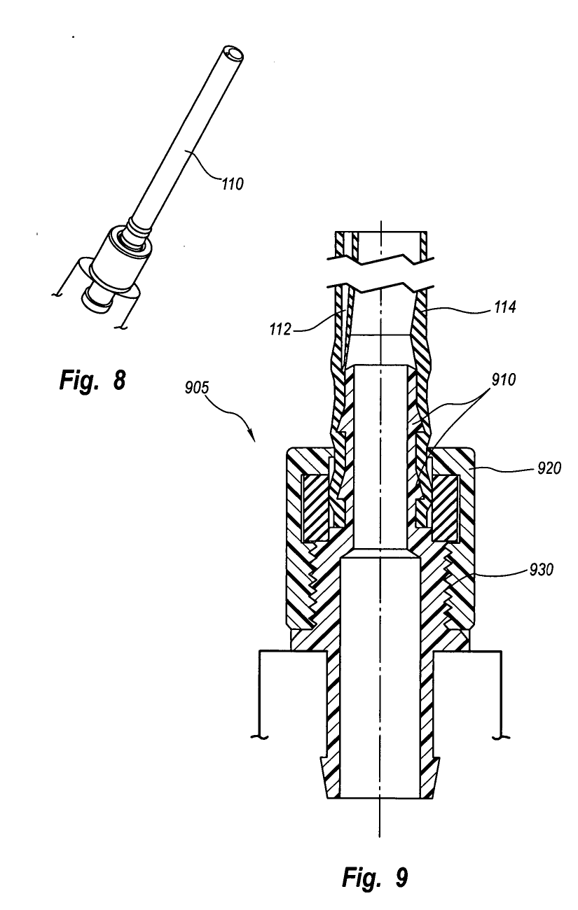

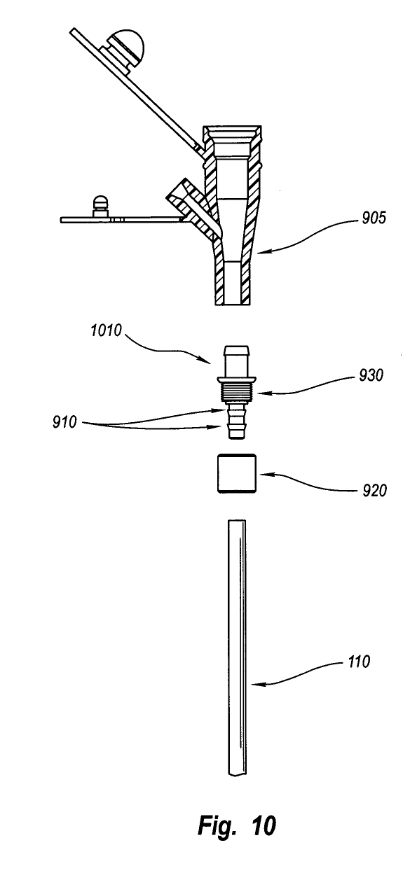

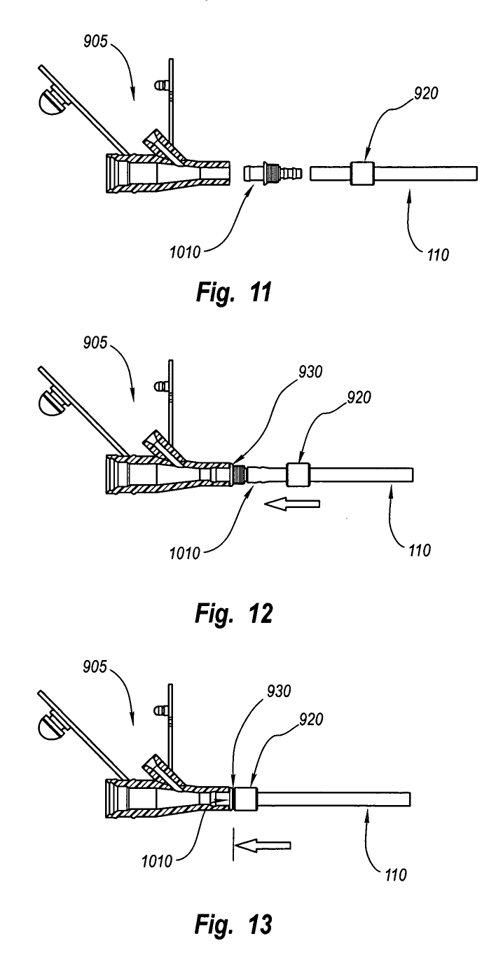

Subsequent to initial placement of the medical device 100, a physician or other person may choose to install a feeding adapter including two lumens or ports, such as feeding adapter 905. Feeding adapter 905 and a method for use thereof is discussed hereinbelow with reference to FIGS. 8-13. Locking cap 920 is placed over the proximal portion of catheter 110 and may be positioned away from the proximal end of catheter 110 as shown in FIG. 11. Then, catheter 110 is slidably positioned over barb fitting 1010 (i.e., barbs 910 thereof) as shown in FIG. 12. Locking cap 920 may be affixed to barb fitting 1010 (e.g., via threads 930) as illustrated by FIG. 13. Also, bolster control lumen 112 may be pinched off or otherwise sealed by compression between the barbed connection (e.g., barbs 910) of feeding adapter 905 and locking cap 920. Accordingly, when clamp device 750 is opened, a pressure within bolster control lumen 112 and internal bolster 140 may be substantially retained. Once feeding adapter 905 is attached to medical device 100, internal bolster 140 and external bolster 760 may be adjusted such that internal bolster 140 is positioned proximate or against stomach wall 302 and external bolster 760 is positioned proximate or against abdominal surface 304.

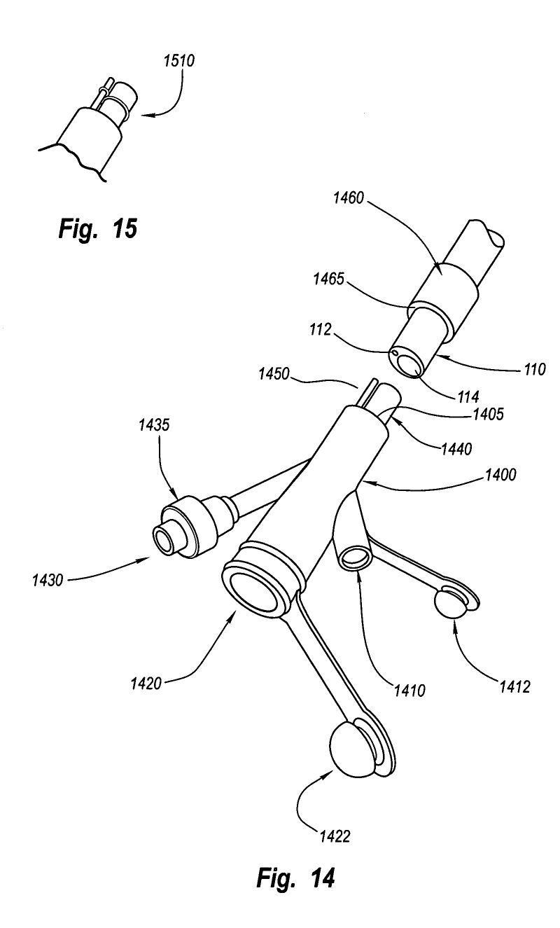

In another embodiment, subsequent to initial placement of the medical device 100, a physician or other person may choose to implement a feeding adapter including three lumens or ports such as feeding adapter 1400 illustrated in FIGS. 14-16. This option may ease performing the expansion and contraction procedure for the internal bolster 140. With respect to embodiments described above, the expansion procedure may be performed before attaching a feeding adapter, and additional equipment may be employed, if necessary, for the deflation procedure (described in more detail below). However, an adapter 1400 may allow a physician to perform the expansion and contraction procedures with the feeding adapter in place. Therefore, medical device 100 may be placed as described above, but the expansion or inflation procedure discussed hereinabove with reference to FIG. 7 may be omitted in favor of the procedures described below.

The following procedure may eliminate the need for some of the components utilized above (e.g., the 3-way stopcock valve, luer-tip needle, and one of the syringes). Additionally, the following procedure may reduce the number of steps and the overall procedure time. More particularly, as described above, medical device 100 may be initially installed using an endoscopic “pull” or “guidewire” procedure. Catheter 110 is cut at cut-line 440 and an external bolster and clamp device as described above may be placed on or coupled to catheter 110. Further, a feeding adapter 1400 (shown in FIG. 14), may include medication port 1410, feeding port 1420, and bolster port 1430. Bolster port 1430 may have a valve element 1435. Valve element 1435 may comprise a check valve of any type as known in the art. Medication port 1410 and feeding port 1420 may have sealing plugs 1412 and 1422, respectively, that are sized and configured for placement within an opening thereof. Subsequent to severing catheter 110, tubing coupler 1460 may be placed thereon, as shown in FIG. 14. Large tube 1440 and small tube 1450 may be inserted or positioned within fistulous lumen 114 and bolster control lumen 112, respectively. Tubes 1440 and 1450 may comprise relatively thin-walled tubes, wherein small tube 1450 may comprise a so-called hypotube, which refers to a stainless steel tube. Tubing coupler 1460 may be advanced along catheter 110, toward adaptor 1400 until clamp face 1465 is positioned proximate to or in contact with face 1405 of adaptor 1400. Tubing coupler 1460 may be sized and configured for compressing the catheter 110 toward tubes 1440 and 1450. Such a configuration may effectively couple catheter 110 to adapter 1400. Also, large tube 1440 may have formed rib 1510 to engage against the interior surface of fistulous lumen 114. This may provide a robust, sealed connection or coupling between adapter 1400 and medical device 100.

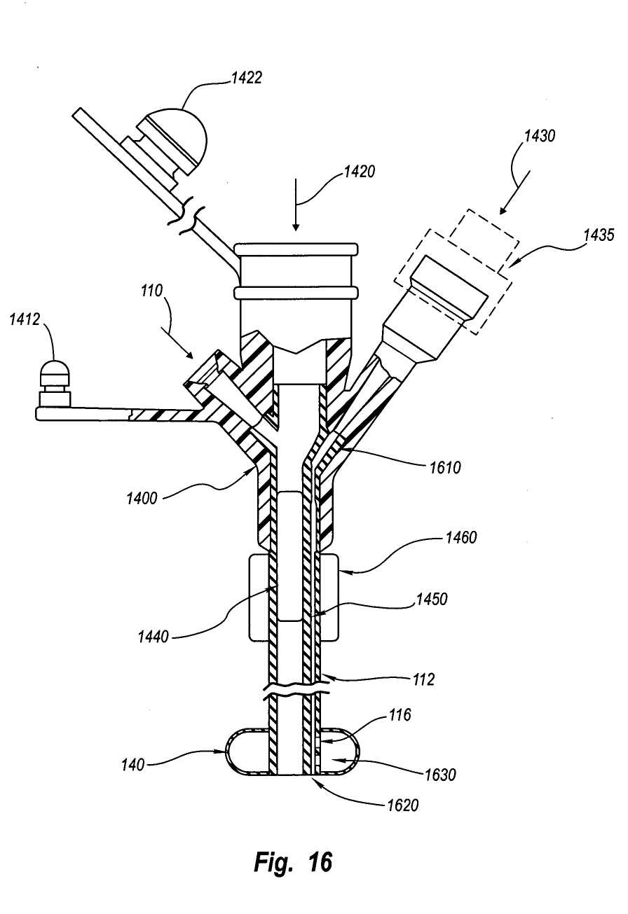

As illustrated by FIG. 16, adapter 1400 may include a rigid insert 1610 to communicate with bolster control lumen 112. Furthermore, bolster control lumen 112 may be sealed by plug 1620 at its distal end. Plug 1620 may comprise an RTV adhesive, silicone, or any other suitable material for sealing the distal end of bolster control lumen 112. In another embodiment, bolster control lumen 112 may be manufactured so as to terminate at bolster control port 116. Internal bolster 140 may be filled with a liquid 1630 (e.g., sterile saline).

Once adapter 1400 has been properly coupled to catheter 110, internal bolster 140 may be expanded or inflated as follows. A syringe, such as syringe 710 of FIG. 7, may be attached to bolster valve 1435 with plunger 712 in the position illustrated in FIG. 7. Plunger control 714 may be operated for removing air, liquid, or both from bolster control lumen 112 and internal bolster 140, if such is present. Holding plunger control 714 in the extended (e.g., rear) position, syringe 710 is disengaged from bolster valve 1435. In addition, another syringe may be filled with the appropriate amount of liquid (e.g., saline), as discussed above with respect to the device's size, and air may be purged from the syringe. The liquid-filled syringe may be attached to bolster valve 1435 and the syringe may be operated to transfer a selected amount of the liquid to internal bolster 140 for expansion or inflation thereof. Subsequent to expansion of internal bolster 140, the syringe may be removed from bolster valve 1435. Also, the internal and external bolsters may be adjusted as described above.

When using medical device 100 as a replacement PEG device, any replacement procedure as known in the art may be employed. Of course, various aspects of such replacement procedures may vary. In one aspect of a replacement procedure, if internal bolster is at least partially expanded, internal bolster 140 may be elongated or otherwise deformed (e.g., by using a stylet or other device) for facilitating insertion of the internal bolster 140 through the existing stoma formed into abdominal surface 304. Once medical device 100 is suitably positioned and internal bolster 140 is positioned proximate the stomach wall 302, the internal bolster 140 may assume its undeformed shape (e.g., by removing the stylet or other deformation device). In another embodiment, medical device 100 may be inserted through the existing stoma with internal bolster 140 in a deflated state, utilizing a deflation notch such as aperture 610 described above. Once internal bolster 140 is positioned at a desired location along stomach wall 302, internal bolster 140 may be expanded (i.e., increased in size) by injecting liquid (e.g., sterile saline) into bolster control lumen 112 using any of the procedures described above in conjunction with a any adapter as disclosed herein or any other suitable adapter.

In another aspect of the present invention, internal bolster 140 may be filled with sterile saline when bolster 140 is intended for securing a medical device within a patient and a vacuum removal procedure may be employed for deflating internal bolster 140 when the medical device is to be removed from a stoma of a patient. Such a configuration may result in superior retention forces (i.e., greater than conventional devices) and improved (i.e., less than conventional devices) removal forces. More particularly, retention and removal forces are illustrated in Table 1. The data compiled in Table 1 was generated in a bench test using a rigid silicone disc with a hole in the center to simulate the stomach wall. A 20 Fr. catheter was used.

While results with human tissue and varying diameter catheters may vary, these results may demonstrate or indicate relative retention and removal forces related to medical devices of the present invention.

TABLE 1

| | | | | | Device According to | |
|---|---|---|---|---|---|---|
| | | | | | the Present Invention | |
| | Conventional Device | | Conventional Device | | Including a Saline- | |
| | with Silicone Solid | | with Air-filled Balloon | | filled Balloon Bolster | |
| Number | Bolster | | Bolster | | Device | |
| of Test | Retention | Removal | Retention | Removal | Retention | Removal |
| Samples | (lbs.) | (lbs.) | (lbs.) | (lbs.) | (lbs.) | (lbs.) |
| 1 | 8.4 | 8.4 | 9.9 | 6.7 | 16.3 | 3.8 |
| 2 | 8.6 | 8.6 | 10.2 | 8.4 | 17.0 | 5.2 |
| 3 | 9.2 | 9.2 | 10.2 | 5.9 | 15.5 | 5.2 |
| 4 | 9.2 | 9.2 | 10.6 | 7.5 | 14.9 | 4.0 |
| Average | 8.6 | 8.6 | 10.2 | 7.1 | 15.9 | 4.5 |
| Std. Dev. | 0.4 | 0.4 | 0.3 | 1.1 | 0.9 | 0.8 |

Referring to Table 1, “retention force” is an amount of force employed for removing a PEG device when the internal bolster is configured for securing the PEG device within a patient (i.e., at least substantially expanded or inflated). Put another way, a retention force is an amount of force employed for prematurely removing the PEG device. On the other hand, “removal force” is an amount of force employed for removing a PEG device when the internal bolster is configured for removal (e.g., substantially deflated) thereof. Both the retention and removal forces may be applied generally perpendicular to the surface of the skin surrounding the stoma and generally parallel to the direction of the incision forming the stoma (i.e., generally aligned with a longitudinal axis of the fistula or catheter 110). Thus, it may be appreciated that a medical device of the present invention may be capable of producing a retention force of at least about 15 lbs. and may be capable of producing a removal force of about 5 lbs. or less. Further, as may be appreciated by considering the data presented in Table 1, a medical device of the present invention may provide a ratio of the retention force to the removal force thereof of about 3 or more (e.g., about 3 to about 4.3).

Conventional devices which incorporate solid internal bolsters such as silicone domes have equal retention and removal forces, because such internal bolsters have an unchanging size. Thus, a removal procedure for a PEG device including a silicone dome bolster may be simply applying force to the proximal end of the device until the silicone dome bolster is deformed sufficiently to allow release of the PEG device from the stoma. Such a removal force may be a force that accidentally or prematurely removes the PEG device or may be applied by a physician or other person to remove the PEG device at the proper time.

As illustrated by Table 1, a conventional air-filled bolster PEG device may exhibit higher retention forces than the conventional solid silicone dome bolster devices. Because the removal procedure includes allowing some of the air to escape from the internal bolster, the air-filled internal bolster may be larger or more rigid in its expanded state than the silicone dome bolster. The feeding tube may have a small airway lumen that is open to the bolster and plugged just proximal of the stoma. The air entrapped in the bolster increases the retention force over the conventional silicone dome bolster initial placement PEG devices. However, due to the compressibility of air, the retention forces may be inconsistent among devices and may result in patient pull out at relatively low retention forces.

Removal forces of a conventional air-filled internal bolster PEG device may also be inconsistent due to the properties of the trapped air and the standard removal procedure. During the standard removal procedure, the feeding tube may be cut distal to a plugged portion of the small airway lumen. This may allow air to evacuate as the bolster is pulled through the stoma. Accordingly, such a procedure may reduce the removal force associated with removing a conventional PEG device including an air-filled internal bolster. However, if the feeding tube is held incorrectly during traction removal, the air lumen may be pinched off, resulting in higher removal forces. Additionally, during the removal step, the bolster wall may fold over the airway notch as the device is pulled through the stoma, sealing off the air lumen and preventing air evacuation. This may result in inconsistent and higher removal forces.

Comparatively, data for a saline-filled internal bolster device was collected with a replacement PEG device in accordance with one embodiment of the present invention. An initial placement device constructed with a similar saline-filled bolster performs in a substantially similar way. Due to the rigidity of an internal bolster filled with liquid (e.g., sterile saline), a substantial increase in retention forces of a PEG device as compared to a conventional PEG device employing an air-filled or solid internal bolster may be exhibited. Such relatively high retention forces may minimize the incident of unintended removal of a PEG device by a patient. Furthermore, vacuum removal of substantially the internal volume of the internal bolster 140, as described in more detail below, may provide a substantial decrease in a removal force as compared to the removal force associated with a conventional PEG device employing an air-filled or solid internal bolster. Such lower removal forces may reduce pain and discomfort which may accompany PEG device removal.

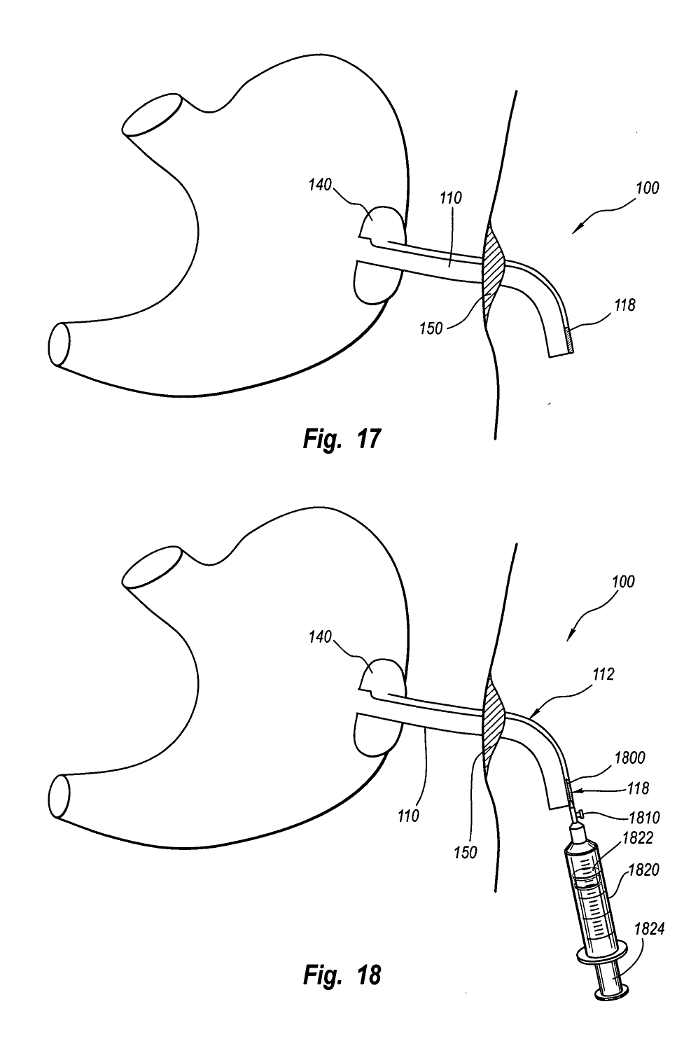

With respect to FIGS. 17-19, a removal procedure for a PEG device according to an embodiment of the present invention is described. FIG. 17 shows medical device 100 positioned in a patient and held therein by internal bolster 140 and external bolster 150. Liquid-filled internal bolster 140 may be sealed by plug 118. Prior to performing the removal procedure, external bolster 760 may be moved away from abdominal surface 304 and clamp device 750 may be removed from catheter 110. FIG. 18 illustrates the attachment of syringe 1820 to medical device 100. Before attaching syringe 1820, catheter 110 may be cut distal to plug 118 such that the proximal end of bolster control lumen 112 is exposed. In embodiments using an adapter, such as 905 discussed hereinabove, the adapter may be cut away at this point in the procedure, in order to expose bolster control lumen 112. Otherwise, an adapter such as adapter 1400, as discussed hereinabove, may be coupled to catheter 110 and may be used to access internal bolster 140, as discussed hereinbelow. Syringe 1820 may be attached to the exposed proximal end of bolster control lumen 112 with luer-tip needle 1800. In another embodiment, plug 118 may be constructed of a material penetrable by luer-tip needle 1800, and luer-tip needle 1800 may simply be inserted through plug 118. In one embodiment, syringe 1820 may have a capability for accepting a volume greater than the volume of liquid within internal bolster 140. Such a configuration may provide an ability to create a relatively robust (i.e., low pressure) vacuum within internal bolster 140.

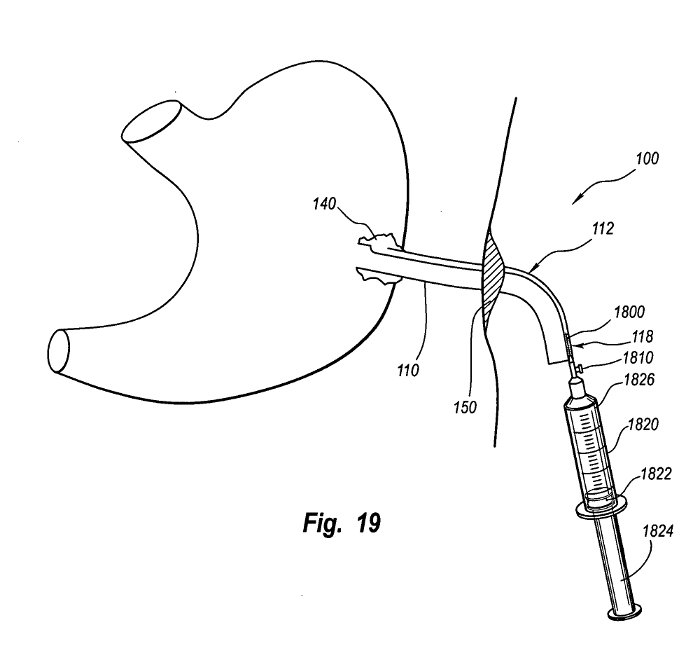

Syringe 1820 may include stopcock 1810. When syringe 1820 is attached to the proximal end of bolster control lumen 112, stopcock 1810 is in the open position and plunger 1822 is in the forward (e.g., distal) position. As shown in FIG. 19, the physician pulls a vacuum with plunger control 1824 to substantially remove liquid, air, or both from internal bolster 140 and bolster control lumen 112. The physician may confirm that an appropriate volume of liquid has been evacuated from internal bolster 140 by verifying that the volume of liquid used to expand internal bolster 140 is now entirely in syringe 1820 including volume markings 1826. The present invention further contemplates that internal bolster may be sealed subsequent to substantially removing the contents (e.g., a fluid) from internal bolster 140. For example, stopcock 1810 may then be closed to hold internal bolster 140 in a collapsed state. Additionally, the physician may install and close a clamp device 750 on catheter 110. Such a procedure may ensure that internal bolster 140 remains substantially deflated and exhibits an associated reduced size (i.e., a collapsed size) during traction removal.

The stoma at abdominal surface 304 may be lubricated for facilitating removal of medical device 100. Further, medical device 100 may be slowly rotated and gently pushed in the distal direction to disengage medical device 100 from the fibrous tract. Abdominal surface 304 may be loosely covered with a towel, drape, or gauze, as may be desired. A physician may grasp medical device 100 proximate to abdominal surface 304 and may apply force thereto, in a direction generally away from the patient, while applying pressure to the abdominal surface 304 generally toward the patient with the other hand. The device may be removed from the patient under such forces, and which may be repeatedly applied by repositioning the pulling hand as the medical device 100 progresses so as to keep it close or near to the abdominal surface 304. During traction removal, the physician may continue to apply pressure to the abdomen with his non-pulling hand. This process is continued until the internal bolster 140 is released from the abdominal wall of the patient.

In another embodiment, the syringe used for removing the contents (e.g., air, liquid, or both) from the internal bolster 140 may have a volume substantially equal to the volume of saline originally used to inflate or expand internal bolster 140. Thus, pulling plunger control 1824 to its proximal most position may remove a liquid from internal bolster 140 and reduce a size thereof. In some embodiments, it may be desired to remove substantially all of the volume of liquid (e.g., sterile saline) so that a relatively low removal force need be applied for removing the medical device 100. The removal procedure outlined with respect to FIGS. 17-19 results in a more consistent force removal, generating lower removal forces than conventional PEG devices.

When an adapter is coupled to catheter 110, such as adapter 1400, the traction removal procedure varies. As above, the external bolster is first positioned away from the abdominal surface. Next, a syringe is attached to the bolster valve. The syringe is operated to evacuate all liquid from the internal bolster, and the syringe is removed. Because of the check valve feature of the adapter 1400, no pinch clamp or stopcock may be employed for causing an internal bolster to remain in its deflated state. The remainder of the traction removal procedure may be substantially the same as described above with respect to the other embodiments.

For safety purposes, medical device 100 may be sterilized with ethylene oxide, e-beam, or Gamma sterilization processes, or any other suitable means of sterilization. The materials used to manufacture medical device 100 and other medical devices in accordance with the present invention may vary. Varying the materials used in medical devices according to the present invention may further improve the retention and removal forces. However, it should be recognized that the internal bolster may be filled with sterile saline or another liquid. Further, substantially removing the volume of liquid within an internal bolster (e.g., by applying a vacuum to an internal chamber of the internal bolster) so as to collapse the internal bolster prior to removal may provide desired retention characteristics and removal forces. The medical device of the present invention has been described through examples of initial placement and replacement PEG devices. However, it should be recognized that the medical device of the present invention may be used to treat other deficiencies and disorders with other body organs (e.g., the bladder).

Thus, it may be appreciated that apparatuses and methods may make it more difficult for a patient to pull out the medical device and reduce the removal forces, which further reduces the pain and discomfort felt by a patient during removal of the device. One skilled in the art will appreciate that the present invention can be practiced by other than the described embodiments, which are presented for purposes of illustration and not of limitation, and the present invention is limited only by the claims which follow. Put another way, while certain embodiments and details have been included herein for purposes of illustrating the invention, it will be apparent to those skilled in the art that various changes in the methods and apparatus disclosed herein may be made without departing form the scope of the invention, which is defined in the appended claims. The words “including” and “having,” as used herein including the claims, shall have the same meaning as the word “comprising.”

1. A method of inserting and removing a medical device from a patient, the method comprising:
positioning a medical device including an internal bolster so as to form a fistula with an organ of a patient, the internal bolster being configured for retaining at least a portion of the medical device within the organ of the patient;
at least partially filling the internal bolster with a liquid;
removing at least a portion of the liquid from the internal bolster; and
removing the medical device from the patient.

2. The method of claim 1, wherein the liquid includes sterile saline.

3. The method of claim 1, wherein removing the medical device from the patient comprises applying a force to the medical device in a direction generally away from the patient; and
contemporaneously with applying the force to the medical device in the direction generally away from the patient, applying pressure to a body surface proximate the medical device in a direction generally toward the patient.

4. The method of claim 1, wherein removing the medical device from the patient comprises applying a force of about 5 lbs. or less to the medical device in a direction generally away from the patient.

5. The method of claim 1, further comprising removing substantially all of the liquid from the internal bolster.

6. The method of claim 5, wherein removing substantially all of the liquid from the internal bolster comprises generating a vacuum within the internal bolster.

7. The method of claim 6, wherein generating the vacuum within the internal bolster comprises generating the vacuum within the internal bolster via a syringe.

8. The method of claim 5, wherein removing substantially all of the liquid from the internal bolster via the syringe comprises operably coupling the syringe to a proximal opening of a lumen in fluid communication with the internal bolster, and operating the syringe to remove substantially all of the liquid from the internal bolster.

9. The method of claim 8, wherein operably coupling the syringe to the proximal opening of the lumen in fluid communication with the internal bolster comprises inserting a luer-tip needle through an occlusion positioned within the proximal opening of the lumen.

10. The method of claim 1, further comprising sealing a lumen in fluid communication with the internal bolster subsequent to removing at least a portion of the liquid from the internal bolster.

11. The method of claim 10, wherein sealing the lumen subsequent to removing at least a portion of the liquid from the internal bolster comprises closing a stopcock.

12. A medical device comprising:
a catheter including a first lumen and a second lumen, the catheter including a distal end and a proximal end; and
an internal bolster attachable to the catheter near the distal end thereof and at least partially fillable with a volume of liquid;
wherein the first lumen is configured for providing a fistula to an organ of a patient and the second lumen is configured for fluid communication with the internal bolster.

13. The medical device of claim 12, further comprising an external bolster coupled to the catheter and movable along a length thereof and configured for, in combination with the internal bolster, retaining at least a portion of the catheter within the patient.

14. The medical device of claim 12, further comprising a syringe connectable with the second lumen, wherein the syringe is configured for removing at least a portion of the liquid and at least partially collapsing the internal bolster.

15. The medical device of claim 14, wherein the syringe is configured for removing substantially all of the volume of liquid from the internal bolster.

16. The medical device of claim 14, wherein the syringe is attachable to the proximal opening of the second lumen with a luer-tip needle.

17. The medical device of claim 12, further comprising a stopcock for selectively controlling fluid communication between the syringe and the second lumen of the catheter.

18. The medical device of claim 12, further comprising an occlusion positionable in a proximal end of the second lumen, wherein the occlusion is self-sealing with respect to needle-penetration.

19. The medical device of claim 12, further comprising an anti-reflux valve operably couplable to the first lumen of the catheter.

20. The medical device of claim 12, further comprising an aperture structured for releasing liquid from the internal bolster.

21. The medical device of claim 12, wherein the internal bolster is configured for providing, upon expansion and contraction thereof, respectively, a retention force of at least about 15 lbs. and a removal force of about 5 lbs. or less.

22. A medical device in the form of a gastrostomy device comprising:
a catheter including a first lumen and a second lumen, the catheter including a distal end and a proximal end;
an internal bolster attachable to the catheter near the distal end thereof;
wherein the proximal end of the catheter includes a pull device configured for facilitating pulling the proximal end of the catheter through a stoma formed in a stomach of a patient; and
wherein the first lumen is configured for providing a fistula to the stomach of the patient, and the second lumen is configured for fluid communication with the internal bolster.

23. The medical device of claim 22, further comprising an external bolster capable of coupling to the catheter and movable along a length thereof and configured for, in combination with the internal bolster, retaining at least a portion of the catheter within the patient.

24. The medical device of claim 22, wherein the pull device comprises a snare loop.

25. The medical device of claim 22, further comprising a syringe connectable with the second lumen, wherein the syringe is configured for removing a volume of liquid from the internal bolster.

26. The medical device of claim 25, wherein the syringe is attachable to the proximal opening of the second lumen with a luer-tip needle.

27. The medical device of claim 26, further comprising a stopcock operably configured for selectively controlling fluid communication between the syringe and the second lumen of the catheter.

28. The medical device of claim 27, further comprising another syringe operably connectable to the stopcock.

29. The medical device of claim 22, further comprising an occlusion positionable in a proximal end of the second lumen, wherein the occlusion is self-sealing with respect to needle-penetration.

30. The medical device of claim 22, further comprising an anti-reflux valve operably connectable to the first lumen of the catheter.

31. The medical device of claim 22, further comprising an aperture formed in the catheter for releasing liquid from the internal bolster.

32. The medical device of claim 22, wherein the internal bolster is configured for providing, upon expansion and contraction thereof, respectively, a retention force of at least about 15 lbs. and a removal force of about 5 lbs. or less.

33. A method of use of a medical device, the method comprising:
providing a catheter including:
a first lumen and a second lumen, the catheter including a distal end and a proximal end; and
an internal bolster attachable to the catheter near the distal end thereof and capable of fluid communication with the second lumen;
wherein the proximal end of the catheter includes a pull device configured for facilitating pulling the proximal end of the catheter through a stoma formed in an organ of a patient and the first lumen is configured for providing a fistula to an organ of a patient;
inserting the distal end of the catheter through an esophagus and into a stomach of a patient;
pulling the distal end of the catheter through a stoma formed in a wall of the stomach so as to position the internal bolster proximate thereto;
severing a portion of the catheter positioned external to the patient; and
at least partially expanding the internal bolster with a liquid so as to facilitate retention of the catheter within the patient.

34. The method of claim 33, further comprising sealing the second lumen subsequent to at least partially expanding the internal bolster.

35. The method of claim 34, wherein sealing the second lumen subsequent to at least partially expanding the internal bolster comprises coupling an adaptor to the catheter.

36. The method of claim 33, wherein at least partially expanding the internal bolster comprises generating a vacuum within the internal bolster and introducing liquid within the internal bolster subsequent to generating the vacuum therein.

37. The method of claim 33, wherein at least partially expanding the internal bolster comprises introducing a liquid therein.

38. The method of claim 37, further comprising:
removing at least a portion of the liquid from the internal bolster; and
removing the catheter from the patient.
